(19) United States
(12) Patent Application Publication
(10) Pub. No.: US 2023/0104711 A1
(43) Pub. Date: Apr. 6, 2023

(54) MULTI-LAYER ABSORBENT PRODUCT AND PROCESS FOR PREPARING ABSORBENT LAYER

(71) Applicant: POLYGREEN LTD., Tel Aviv (IL)

(72) Inventors: Shai SHAHAL, Tel Aviv (IL); Zvi MEIRI, Netanya (IL); Uri ASH, Kahal (IL); Peter TEPLITZKY, Haifa (IL)

(21) Appl. No.: 17/904,605

(22) PCT Filed: Feb. 20, 2021
(86) PCT No.: PCT/US2021/018938

§ 371 Date: Aug. 19, 2022

Related U.S. Application Data
(60) Provisional application No. 62/979,261, filed on Feb. 20, 2020.

(51) Int. Cl. A61L 15/24; A61L 15/60; C08L 23/12

(52) U.S. Cl. CPC A61L 15/24; A61L 15/60; C08L 23/12; C08L 2203/12

(57) ABSTRACT

A multi-layer absorbent product is provided that includes a first layer that is either liquid permeable or liquid impermeable and a layer of a treated nonwoven, woven or knitted textile material or a treated textured plastic yarn. The layer of textured plastic yarn or of nonwoven, woven or knitted textile material has uniformly spaced deposits of a natural polymer crosslinked to a synthetic polymer in the absence of non-polymeric crosslinking agent, where each of the deposits is a continuous polymer network that covers the textile or plastic underneath the deposit and interpenetrates the network of textile fibers or of plastic pores acting as a scaffold to interconnect the polymer network with the fiber network or with the network of plastic pore walls. A process for preparing the layer of textile material or of textured plastic yarn with uniformly spaced deposits is also provided.

### FIELD OF THE INVENTION

The present invention relates to a process for production of water-absorbing textile material such as non-woven, woven or other textile materials types or water-absorbing plastic yarn with textured surface and related absorbing products.

### DESCRIPTION OF THE RELATED ART

In art there are known processes to manufacture an absorbent textile material, which can be divided in two distinct classes: a) processes with textile fibers and polymeric absorbents, b) processes with prefabricated textile materials and reactive liquid media.

Processes with Prefabricated Textile Materials and Reactive Liquid Media

Particularity of these processes consists in that, a traditional prefabricated textile material like nonwoven, woven, knitted or braided type, is impregnated with a fluid mass in solution, emulsion or suspension form, and a polymeric absorbent is generated in situ during the drying of the impregnated wet textile material, or after drying and applying of an adequate thermal treatment. Representative processes that are known in the art: b1) processes with polymerizable impregnation mass and b2) processes with cross-linkable impregnation mass.

Processes with Polymerizable Impregnation Mass

In these processes, a liquid used for impregnation produces in-situ polymerization of partially neutralized acid vinyl monomers directly on a synthetic nonwoven substrate. These processes can be found in U.S. Pat. Nos. 4,537,590; 4,540,454; 4,573,988; 4,676,784; 5,567,478; 5,962,068; 6,417,425 and 6,645,407.

Processes with Cross-Linkable Impregnation Mass

In these processes, the textile material prefabricated as: nonwoven, woven or other known type is impregnated with a fluid mass in the form of solution or emulsion, that contains polymers in dissolution or dispersion state, or in mixture with auxiliary materials which are cross-linking agents active at temperatures and induce generation in situ of a polymeric absorbent. In this class can be seen more variants:

i) Processes with Impregnation Mass that Contain a Self-Crosslinking Synthetic Polymer.

Various processes are described in which a substantially linear acrylic polymer is cross linked through its pendant groups. The pendant groups will react with each other upon appropriate heating. Any combination of monomers that will undergo such reaction can be used as in U.S. Pat. Nos. 4,057,521; 4,861,539; 4,962,172; 4,963,638; 5,280,079 and 5,413,747.

The impregnation mass based on self-crosslinking polymers has the disadvantage that the cross-linking is not complete and because of this reason more than 10% of end product is lost during processes associated with extraction of monomers, initiators and organic solvents residue used during polymerization. Moreover, the free absorbency of the resulting cross-linked polymers has a low value (less than 50 g/g in 0.9% NaCl solution), and the product is not safe.

ii) Processes with Impregnation Mass that Contain Synthetic Polymers and Cross-Linking Agents

It is further known from U.S. Pat. Nos. 2,988,539; 3,393,168 and 3,514,419 that water swellable cross-linked carboxylic copolymers can be prepared. However, these prior art copolymers are all cross-linked during copolymerization or cross-linked after polymerization with subsequent neutralization of the carboxylic acid groups to form water swellable polyelectrolytes and hence these prior art polyelectrolytes cannot be cross-linked in situ as a coating on a substrate or as a flexible film thereof.

U.S. Pat. Nos. 3,926,891; 3,980,663 and 4,155,957 present chemical structures of the principal classes of combinations that can be used as cross-linking agents for polymers with free carboxylic chemical functions. Also is shown that cross-linking reaction occurs based on mechanism known as nucleophilic displacement on saturated carbon. The carboxylate ion on polymer acts as nucleophile while cross-linking agent is the substrate for nucleophilic attack.

Cheng et al. in U.S. Pat. No. 5,693,707 presents an aqueous polymer composition comprising 10 to 40 wt % of a polymer in water, the polymer consisting essentially of 20-90 wt % alpha, beta-ethylenically unsaturated carboxylic acid monomer, 10-80 wt % one or more softening monomers, the aqueous composition being adjusted to pH 4-6 with alkali metal hydroxide or alkaline earth metal hydroxide and further containing 0.1 to 3 wt % zirconium crosslinking salt. Such aqueous compositions are applied to nonwoven and woven substrates to make absorbent textile web.

Goldstein et al. in U.S. Pat. No. 6,506,696 shows that the method of forming the high performance nonwoven webs of this invention comprises: applying an aqueous polymeric emulsion containing a polymer having dual cross-linkable functionality to a synthetic based nonwoven web, wherein the dual cross-linkable polymer incorporates acetoacetate functionality and carboxylic acid functionality; removing water and cross-linking the cross-linkable polymer with an effective amount of a polyaldehyde and an effective amount of a polyaziridine compound.

Soerens Dave Allen in U.S. Pat. No. 7,205,259 presents an absorbent binder desiccant composition which is capable of spontaneous cross-linking after application to a substrate, at a temperature of about 120° C. or less. The absorbent binder desiccant composition includes a monoethylenically unsaturated polymer, such as carboxylic acid, sulphonic acid, or phosphoric acid, or salts thereof, or a quaternary ammonium salt, and an acrylate or methacrylate ester that contains an alkoxysilane functionality, or a monomer capable of co-polymerization with a compound containing a trialkoxy silane functional group and subsequent reaction with water to form a silanol group, and a desiccant component. The absorbent binder desiccant composition is particularly suitable for use in manufacturing a wide variety of humidity control articles.

A diversity of chemical compositions (polymers and cross-linking agents) besides of multiple variants of control of cross-linking reactions are found in art in U.S. Pat. Nos. 3,983,271; 4,066,584; 4,320,040; 4,418,163; 4,731,067; 4,855,179; 4,880,868; 4,888,238; 5,698,074; 5,997,791; 6,150,495; 6,162,541; 6,241,713; 6,773,746 and 6,824,650.

iii) Processes with Impregnation Mass that Contain Biopolymers and Cross-Linking Agents

Weerawarna et al. in U.S. Pat. No. 7,300,965 provides a mixed polymer network having superabsorbent properties. The composition is obtainable by reacting a carboxyalkyl cellulose and a synthetic water-soluble polymer having carboxylic acid or carboxylic acid derivative substituents with a crosslinking agent. The cross-linking agent reacts with at least one of the carboxyalkyl cellulose or water-soluble polymer to provide the network. Suitable cross-linking agents include cross-linking agents that are reactive toward carboxylic acid groups.

Representative organic cross-linking agents that are reactive toward carboxylic acid groups include diols and polyols, diamines and polyamines, diepoxides and polyepoxides, polyoxazoline functionalized polymers, and aminols having one or more amino groups and one or more hydroxy groups.

Representative inorganic cross-linking agents that are reactive toward carboxylic acid groups include polyvalent cations and polycationic polymers. Exemplary inorganic cross-linking agents include aluminum chloride, aluminum sulfate, and ammonium zirconium carbonate with or without carboxylic acid ligands such as succinic acid (dicarboxylic acid), citric acid (tricarboxylic acid), and butane tetracarboxylic acid (tetracarboxylic acid). Water soluble salts of trivalent iron and divalent zinc and copper can be used as cross-linking agents. Representative carboxylic acid cross-linking agents includes di- and polycarboxylic acids. U.S. Pat. Nos. 5,137,537; 5,183,707 and 5,190,563 describe the use of C2-C9 polycarboxylic acids that contain at least three carboxyl groups (e.g., citric acid and oxydisuccinic acid) as crosslinking agents. Suitable polycarboxylic acid crosslinking agents include citric acid, tartaric acid, malic acid, succinic acid, glutaric acid, citraconic acid, itaconic acid, tartrate monosuccinic acid, maleic acid, 1,2,3-propane tricarboxylic acid, 1,2,3,4-butanetetracarboxylic acid, all-cis-cyclopentane tetracarboxylic acid, tetrahydrofuran tetracarboxylic acid, 1,2,4,5-benzenetetracarboxylic acid, and benzenehexacarboxylic acid. The cross-linking can be achieved by heating at a temperature and for a period sufficient to effect the cross-linking. The carboxymethyl cellulose solution containing cross-linking agent or synthetic water-soluble polymer and cross-linking agent can be air-dried or solvent precipitated followed by cross-linking. Cross-linking time and temperature will depend on the cross-linking agent and polymers used.

Sun et al. in U.S. Pat. No. 6,689,378 presents methods of immobilizing uncomplexed cyclodextrins and complexed cyclodextrins to polysaccharide containing substrates, such as cellulose fibers by covalently bonding. The cellulose/cyclodextrin compositions can be used in all types of cellulose fiber containing articles, such as tissues and personal care articles.

Useful polymeric anionic reactive compounds are compounds having repeating units containing two or more anionic functional groups that will covalently bond to hydroxyl groups of the substrate. Exemplary polymeric anionic reactive compounds include the ethylene/maleic anhydride copolymers described in U.S. Pat. No. 4,210,489. Vinyl/maleic anhydride copolymers and copolymers of epichlorohydrin and maleic anhydride or phthalic anhydride are other examples. Copolymers of maleic anhydride with olefins can also be considered, including poly(styrene/maleic anhydride)

Copolymers and terpolymers of maleic anhydride that could be used are disclosed in U.S. Pat. No. 4,242,408.

The cross-linkable impregnation mass, similar to polymerizable systems in situ have disadvantage that the cross-linking is not complete in conditions of thermal treatment mentioned, existing permanent the possibility that during uses of textile products to extract besides soluble polymer also cross-linking agent, this being more dangerous for the human healthy comparatively with the monomers or polymerization auxiliary residues. Presence of biopolymers in mixture for treating of textile materials, not contribute to improving the chemical transformation yield that occurs during cross-linking Referring to uses of macromolecular cross-linking agents, these products being in their turn result of synthesis processes are not mentioned their purity or the extractability in aqueous solutions used as swelling media.

A particular drawback of known processes in the art for obtaining absorbent textiles is that those products are not ecological. Majority of polymeric absorbents discussed above, are products of synthesis, which because of their chemical structure have not the capacity of biodegradation in specific active biological media, i.e. the domestic compost. Moreover, absorbent textiles that contain biopolymers, although are biodegradable, have small values of free absorbency. Another drawback of known absorbent textiles is that the polymer network included in such textiles was obtained through the use of crosslinking agents that might present health hazards to end users.

Citation of any document herein is not intended as an admission that such document is pertinent prior art or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention eliminates the disadvantages of processes known in art for obtaining absorbent textile products or absorbent textured plastic yarn products by providing a new process for treating a textile material or textured yarn as substrate and using the treated textile material or textured yarn with improved water absorbance and biodegradability as the basis to form a multi-layer absorbent product. The new process makes use of an aqueous textile-impregnating or textured yarn-impregnating polymeric composition in the form of a syrup (containing a high concentration, e.g., at least 20%-40%, of polymers in the solution) of at least two types of soluble polymers—a synthetic polymer and a natural polymer, with the syrupy solution being devoid of separate non-polymer crosslinking agent. The syrup of soluble polymers is deposited on the textile material or on the textured yarn in a uniformly spaced manner such that, when the textile material or the textured yarn with uniformly spaced deposits thereon is exposed to thermal/heat treatment, the polymers in the deposits impregnate the textile material or the textured surface of the plastic yarn and interpenetrate into the network of textile fibers of the textile material or the textured network of the plastic yarn and undergo self-crosslinking of the natural and synthetic polymer in the deposits.

The present invention further provides a multi-layer absorbent product for use in absorbent products such as diapers, feminine hygiene absorbent pads, panty liners, cleaning wipes, household or institutional cleaning or maintenance appliances, hand wipes, hand towels, personal, cosmetic or sanitary wipes, baby wipes, facial tissues, hygienic absorbent pads, panties, wound dressings and the like. This multi-layer absorbent product includes a first layer that is either liquid permeable or liquid impermeable (e.g., a bottom layer) and a layer of a treated nonwoven, woven or knitted textile material disposed next to and against the first layer. The layer of nonwoven, woven or knitted textile material has on top uniformly spaced deposits of a natural polymer crosslinked to a synthetic polymer in the absence of non-polymeric crosslinking agent. Each of the deposits of crosslinked natural and synthetic polymers is a continuous polymer network that covers the textile underneath the deposit and interpenetrates the network of textile fibers acting as a scaffold to interconnect the polymer network with the fiber network. The multi-layer absorbent product is also characterized by the high biodegradability of the deposits.

The present invention further provides a multi-layer absorbent product for use in the outdoor coverage or packing or packaging of vegetal agricultural crops such as fodder, hay or cotton, or crop residues such as straw, and their protection from rain, snow or ground moisture and the resultant damage from such uncontrolled humidity, to allow for their storage outdoors. This multi-layer absorbent product includes a first layer that is either liquid permeable or liquid impermeable (i.e., an upper layer) and a layer of a treated plastic yarn with textured surface, disposed next to, below and against the first layer. The layer of textured plastic yarn has on top uniformly spaced deposits of a natural polymer crosslinked to a synthetic polymer in the absence of non-polymeric crosslinking agent. Each of the deposits of crosslinked natural and synthetic polymers is a continuous polymer network that covers the textured yarn underneath the deposit and interpenetrates the network of textured plastic material acting as a scaffold to interconnect the polymer network with the plastic texture network. The multi-layer absorbent product is also characterized by the high biodegradability of the deposits.

### BRIEF DESCRIPTION OF THE DRAWINGS

The invention will now be described with reference to the drawings, wherein:

FIG. 1 is a graph showing the correlation between the viscosity of SP-NP composite solutions, the degree of neutralization of the synthetic polymer (SP) and solution stability. The SP is represented by SMAC (Styrene Maleic Acid Copolymer) in salt form following neutralization with sodium hydroxide. The natural polymer (NP) is represented by gelatin. The following polymer solutions were tested: 100% SMAC; 90% SMAC and 10% Gelatin; 70% SMAC and 30% Gelatin. The SMAC used had different degrees of neutralization as further explained in Example 1. From FIG. 1 it is noted that the presence of the natural polymer Gelatin leads to a decrease in the viscosity of the polymer composite solution if the degree of neutralization of the synthetic polymer is between 30-60%. If the degree of neutralization is greater than 60%, the presence of the natural polymer causes an increase in the viscosity value of the composite solutions in comparison to same solution without natural polymer. With reference to the stability of the composite polymer solutions, it is noted that the composite solutions containing SMAC and gelatin are stable if the degree of neutralization of the synthetic polymer SMAC is greater than 30%.

FIG. 2 is a graph showing the influence of thermal treatment to which is subdue a non-woven sample impregnated with the polymer composite solution containing synthetic polymer and gelatin on the relative absorbency RQ1.

FIG. 3 is a graph showing the influence of the type of fibers on water absorbency for textile impregnated with the polymer composite vs. a textile non-impregnated.

FIG. 4 is a schematic illustration of the process that occurs between a sample of textile material or a textured plastic tarn and the polymer network deposited thereon under heating conditions to provide the textile composite material or the yarn composite where the polymer network interpenetrates the textile fiber network or the plastic surface pores. On the left, (1) presents a schematic illustration of a textile material, illustrating the fibers in small scale (top), large scale top-view (center) and large scale side view (bottom), where few layers of fibers can be seen, with dots representing in-plane fibers. In the middle (2), the process of roll printing is illustrated where the polymer solution is fed from a container into a perforated roll, from which it is deposited on the layer of textile material or plastic yarn; the dots on the layer represent the circular deposits. On the right (3), the finished composite including the substrate textile layer and dried, heat-treated polymer is illustrated: small scale (top) showing deposits larger than fibers and covering them, large scale top-view (center) showing one deposit on the fibers, and large scale side-view (bottom), illustrating the interpenetration of polymer in between the fibers to some depth, wrapping the fibers underneath the deposit, where this network of polymer and fiber or pore-walls composite act as a scaffold that holds the deposited polymer in place, on top of the substrate layer.

FIGS. 5A and 5B show uniformly spaced deposits of a concentrated solution of a natural polymer and a synthetic polymer on textile material, where before thermal treatment and self-crosslinking of the natural and synthetic polymers the deposits are half dome in shape (FIG. 5A) and become circular dots after thermal treatment (FIG. 5B, on a different textile material from FIG. 5A). FIG. 5C shows rectangular shaped deposits after thermal treatment.

FIGS. 6A and 6B are schematic illustrations showing a side view (not to scale) of two configurations of a two-layer absorbent product of the invention, where one of the two layers is a layer of nonwoven, woven or knitted textile material or textured plastic yarn with deposits of superabsorbent polymers (SAP) on one side and the other layer is a liquid permeable or liquid impermeable layer. The arrows represent the direction of liquid flow/penetration.

FIGS. 7A and 7B are schematic illustrations showing a side view (not to scale) of two different embodiments with two layers (FIG. 7A) or three layers (FIG. 7B) of nonwoven, woven or knitted textile material or textured plastic yarn with deposits of superabsorbent polymers (SAP) in the multi-layer absorbent product, which may have an additional layer(s) of a liquid permeable layer and/or a liquid impermeable layer (not shown) as an outer layer or layers of the product. The arrows represent the direction of liquid flow/penetration.

### DETAILED DESCRIPTION OF THE INVENTION

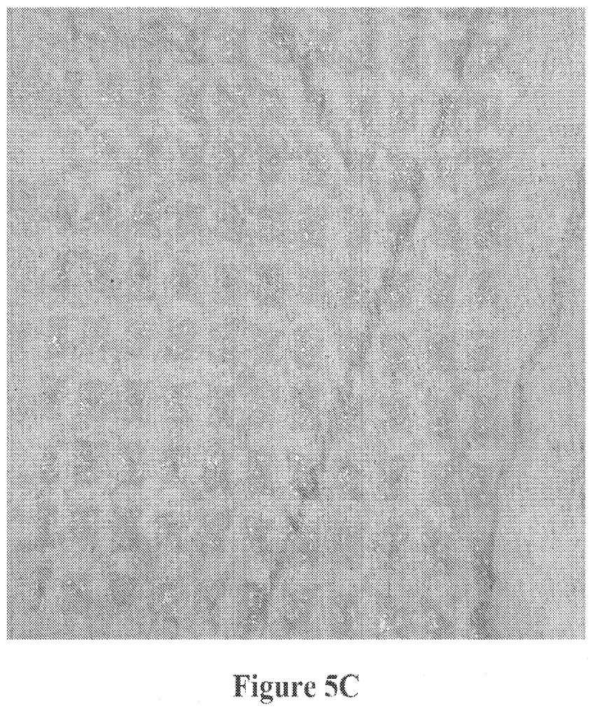

The present invention provides a multi-layer absorbent product composed of at least a “first” layer that is either liquid permeable or liquid impermeable layer (such as the bottom sheet/layer in a diaper or feminine hygiene pads or liners) and a layer of textured plastic yarn or nonwoven, woven or knitted textile material composed of a network of fibers which is disposed next to and against the first liquid permeable or impermeable layer. A layer of nonwoven, woven or knitted textile material has an upper porous surface of fibers on which uniformly spaced deposits of a continuous polymer network of natural polymer crosslinked to a synthetic polymer (in the absence of non-polymeric crosslinking agent) cover the upper fibers of the textile material underneath each deposit and interpenetrates the network of fibers acting as a scaffold. A layer of textured plastic yarn also has an upper porous surface, but one created by non-penetrating holes, indentations, depressions, open bubbles, and other roughness-creating features formed by foaming, etching, cutting or other void-shaping methods. The depth of this surface porosity network should be sufficient to act as a scaffold for uniformly spaced deposits of a continuous polymer network of natural polymer crosslinked to a synthetic polymer (in the absence of non-polymeric crosslinking agent) that interpenetrate the surface pores underneath each deposit. This interpenetration of the network of fibers or plastic pores by the continuous polymer network interconnects the polymer network with the textile fiber network or with the surface pores in the plastic yarn, respectively. See, for instance, the schematic illustration in FIG. 4, where the continuous polymer network interpenetrates the network of textile fibers or network of pore-walls of the textured plastic yarn, interconnecting the two networks. Each of the uniformly spaced deposit covering the textile material directly underneath (impregnating and also interpenetrating the network of fibers underneath) is shown in FIG. 5B. Visually, the deposit appears to be “stuck” or “glued” to the textile material underneath. This provides a “glue-less” way to stick the deposit tightly to fiber network of the textile material substrate underneath, without any danger of the deposit falling apart or falling off because of vibration and speed (sheer forces) during production. The top surface of the layer of nonwoven, woven or knitted textile material with the uniformly spaced deposits is positioned next to and against the first liquid permeable or impermeable layer. The dry uniformly spaced deposits on one surface (e.g., top surface) of the layer of nonwoven, woven or knitted textile material confers to the impregnated textile superior free absorbance when exposed to aqueous fluid such as body fluids. When the impregnated textile with deposits thereon is exposed to aqueous liquids, it has sufficient free spaces that permit fast access and absorption of the liquid.

The density of the nonwoven, woven or knitted textile material (without the deposits), serving as substrate for the deposition/application of a thick and sticky syrup of natural and synthetic polymers (i.e., prior to being heat treated to self-crosslink the natural and synthetic polymers into a network of superabsorbent polymers (SAP)) is in a range between 20 to 150 g/m², preferably 20 to 60 g/m², more preferably 20 to 30 g/m². The textile material as substrate for the deposits of superabsorbent polymers (SAP) that make up the continuous polymer network include prefabricated textile materials such as nonwoven, woven or any other type known in art and commercially available, that are formed from synthetic fibers or natural fibers or a mixture of synthetic and natural fibers. The textile material may be pre-treated with additives or subject to surface treatment with chemicals that may confer on the fabric additional characteristics such as hydrophilicity, hydrophobicity or any other characteristics formed by processes known in the art of textile manufacture. Non-limiting examples of fibers in the textile material as carrier/substrate include synthetic fibers such as polypropylene (PP), polyester, polyvinyl alcohol, etc., and natural fibers such as polylactic acid (PLA), cellulose, viscose, bamboo, cotton, wool, paper, etc. The textile material may be biodegradable by itself, such as for example when made of PLA, or may become biodegradable when dry deposits of SAP are activated by contact with aqueous biological media, such as bodily fluids, to swell and transform into a gel-like material within the textile and on its surface. Preferably, the textile material as substrate is a nonwoven. A nonwoven PP textile material is a preferred embodiment of the substrate. Preferably, the textile material is thin so as to have the benefit of making the multi-layer absorbent product thinner than conventional, i.e., appears more two dimensional (2D) than the conventional fluffy media, which is three dimensional (3D).

The terms fabric, textile, web, with or without being combined with the word “material” are used in this specification interchangeably.

The thickness of the plastic yarn (without the deposits), serving as substrate for the deposition/application of a thick and sticky syrup of natural and synthetic polymers (i.e., prior to being heat treated to self-crosslink the natural and synthetic polymers into a network of superabsorbent polymers (SAP)) is in a range between 20 to 200 micrometers, preferably 20 to 80 micrometers. The density of the plastic yarn is in a range between 18 and 150 gr/m², preferably 18 to 77 gr/m². The plastic yarn as substrate for the deposits of superabsorbent polymers (SAP) that make up the continuous polymer network include prefabricated plastic yarn that may be produced by extrusion or any other method known in art and commercially available, made from synthetic polymers or natural polymers. Non-limiting examples of plastic materials that can form a carrier/substrate yarn include synthetic polymers such as polyethylene (PE), polypropylene (PP), polyester, polyvinyl alcohol, etc., and natural polymers such as polylactic acid (PLA) and cellulose. The plastic yarn may not be biodegradable, or may be biodegradable by itself, such as for example when made of cellulose, or may become biodegradable when dry deposits of SAP are activated by contact with water, to swell and transform into a gel-like material within the plastic yarn and on its surface. The plastic yarn may also be biodegradable following a prolonged weathering process such as exposure to light and air, or may be non-biodegradable following such treatment. Preferably, the plastic yarn as substrate is made of polyethylene (PE), which is a preferred embodiment of the substrate.

The weight of the deposits relative to the textile material or the plastic yarn as substrate/carrier is in a range of 100% to 600%, preferably 200% to 600%, more preferably 400% to 600%. This is the percent add-on weight to textile material substrate/carrier, where, for example, if a nonwoven textile material substrate/carrier is 22 grams per square meter (g/m² or gsm) with 88 gsm of the dry deposit (of SAP) deposited on the substrate/carrier, then the total weight is 110 gsm and the % add-on weight is 400%. Table 5 in Example 17 below shows an example of a textile material substrate/carrier where the add-on weight of the SAP deposited/printed on the nonwoven substrate is 175%. The textile material with the SAP deposits is still safe when in contact with the human body and is environmentally friendly, conferring a pronounced ecologic character to textile materials used.

The textile material with deposits of SAP as a superabsorbent layer in the multi-layer absorbent product has a water absorbency (free absorbance capability) in the range of at least 10% (e.g., at least 10%, 20%, 30%, 40%, 50%, 60%, 70%, 80%, 90%, or 100%) to 10 times higher than the absorbency of the same layer without the deposits (i.e., the non-impregnated textile material substrate), preferably at least 2 times higher than the absorbency of the same layer without said deposits, preferably at least 3 or 4 times, more preferably at least 5 or 6 times, most preferably at least 10 times.

On another measure of absorbency, the textile material or textured plastic yarn with deposits of SAP as a superabsorbent layer is capable of absorbing in the range of at least about 2 to 100 grams water per gram (g/g; gr/gr) on the basis of the layer, such as at least 3 gr/gr, at least 4 gr/gr, at least 5 gr/gr, at least 6 gr/gr, at least 7 gr/gr, at least 8 gr/gr, at least 9 gr/gr, at least 10 gr/gr, preferably at least 20, 30, 40, or 50 gr/gr, more preferably at least 60 gr/gr, 70 gr/gr, 80 gr/gr, or 90 gr/gr, most preferably at least 100 gr/gr.

A preferred embodiment of the dry deposit of SAP on the textile material substrate or plastic yarn substrate is where about 10% (e.g., ±1%) of the SAP deposited on the substrate interpenetrate the network of fibers (e.g., tangle of fibers in a nonwoven), e.g., at least partially penetrating, within the textile material substrate or within the plastic yarn pores and the remainder (about 90%±1%) of the SAP is stuck to the upper porous surface of the textile substrate or the textured plastic yarn substrate to form a continuous SAP crosslinked polymer network that interconnects with the fiber network or network of pores (by sticking to and also interpenetrating the fiber network or the network of pores). Because SAP needs room to swell with water to take advantage of its superabsorbent properties and because, unlike pulp fibers that are loose and have space to swell with water, the fibers in nonwoven textile material used as substrate are tangled to each other in such a way that only allow for limited swelling (“blowing up”) within the network of tangled fibers, the interpenetration of SAP into the fiber network is such that the space for swelling within the network of fibers is not limiting for the amount of SAP, e.g., about 10%, that interpenetrate the network of fibers. The SAP in the deposit that is stuck to the upper porous surface of the textile substrate or plastic substrate however is not constrained by space to swell.

The deposits on the textile material substrate as the layer of nonwoven, woven or knitted textile material are uniformly spaced with a spacing in a range of 1 to 7 mm, preferably 1 to 6 mm, more preferably about 2 mm.

Preferably, the uniformly spaced deposits on the textile material substrate are in the shape of circular dots or rectangles (FIGS. 5B and 5C) but could be any shape or color that may be desirable, such as from a functional or aesthetic perspective. The size of the deposits is such that the space in between the deposits allows for fast penetration of liquids (such as that of water or body fluids, e.g., urine, menstrual fluid, etc.) for contacting the deposits on the first layer of textile material substrate or plastic yarn substrate in which the liquids comes into contact and then allowing some of the excess liquid not quickly absorbed by the first layer to move on to the deposits on any additional subsequent layers (in the direction of liquid flow or penetration) of textile material substrate or plastic yarn substrate in the multi-layer absorbent product.

In the deposits of SAP from crosslinked natural and synthetic polymers (crosslinked in the absence of non-polymeric crosslinking agents), the synthetic polymer has monomers bearing carboxylic acid or carboxylic acid anhydride groups and is preferably selected from linear or branched graft homo- or copolymers made from vinyl acidic monomers such as acrylic acid, maleic anhydride, itaconic anhydride and similar, optionally in association with other types of vinylic monomers that do not necessarily contain carboxylic acid functions. More preferably, the synthetic polymer is a copolymer based on maleic anhydride and/or maleic acid, preferably a copolymer of styrene maleic anhydride (SMA), copolymer of isobutylene and maleic anhydride (e.g., commercially available copolymers sold under the tradename ISOBAM™) or copolymer of methyl vinyl ether and maleic acid (e.g., commercially available copolymers sold under the tradename GANTREZ™).

Also in the deposits of SAP from crosslinked natural and synthetic polymers (crosslinked in the absence of non-polymeric crosslinking agents), natural polymer (NP) is a biopolymer selected from polysaccharides such as cellulose, alginate, dextran, chitosan, and the like; bio-polyesters and lignin (in native forms or modified by chemical or enzymatic hydrolysis); guar; starch; water soluble phospholipids such as lecithin; polypeptides or proteins such as gelatin, albumin, soybean protein, collagen, collagenic biopolymer, collagen hydrolysate, and casein. The natural polymer has amino and/or hydroxyl groups capable of cross-linking to COOH groups in said synthetic polymer under high temperature conditions and for selected periods of time. The natural polymer is a biopolymer that functions as both cross-linking agent of the synthetic polymer and as activator of biodegradation processes. Thus, the natural polymer activates the biodegradation of the synthetic polymer and confers to the system superior biodegradability.

The natural polymer (NP) is preferably a commercially available biopolymer which belong to the following classes of substances: proteins, carbohydrates, bio-polyesters or lignin (as native forms or modified by chemical or enzymatic hydrolysis); preferably the proteins and carbohydrates are soluble in water and have the following characteristics:

a) The average molecular weight from 5 to 100 kDa, preferably from 25 to 50 kDa, more preferably from 75 to 100 kDa

b) Free chemical functions as NH₂; OH—CH₂; OH—C₆H₃₋₅; as single type or more types which have content from 0.001 to 0.01 mol/grams.

The multi-layer absorbent product based on textile material substrate layers according to the invention is for use in absorbent products such as diapers, feminine hygiene absorbent pads, panty liners, cleaning wipes, household or institutional cleaning or maintenance appliances, hand wipes, hand towels, personal, cosmetic or sanitary wipes, baby wipes, facial tissues, hygienic absorbent pads, panties, wound dressings and the like. Where more absorbent layers are needed, such as in diapers, more than one layer of the textile material with uniformly spaced deposits of SAP thereon may be used in the multi-layer absorbent product. For instance, where desirable, there may be 2-5 layers in the multi-layer absorbent product.

The multi-layer absorbent product based on textured plastic yarn layers according to the invention is for use in in the outdoor coverage or packing or packaging of vegetal agricultural crops such as fodder, hay or cotton, or crop residues such as straw, and their protection from rain, snow or ground moisture and the resultant damage from such uncontrolled humidity, to allow for their storage outdoors. Where more absorbent layers are needed, such as to mitigate heavy rainfall, more than one layer of the textured plastic yarn with uniformly spaced deposits of SAP thereon may be used in the multi-layer absorbent product. For instance, where desirable, there may be 2-5 layers in the multi-layer absorbent product.

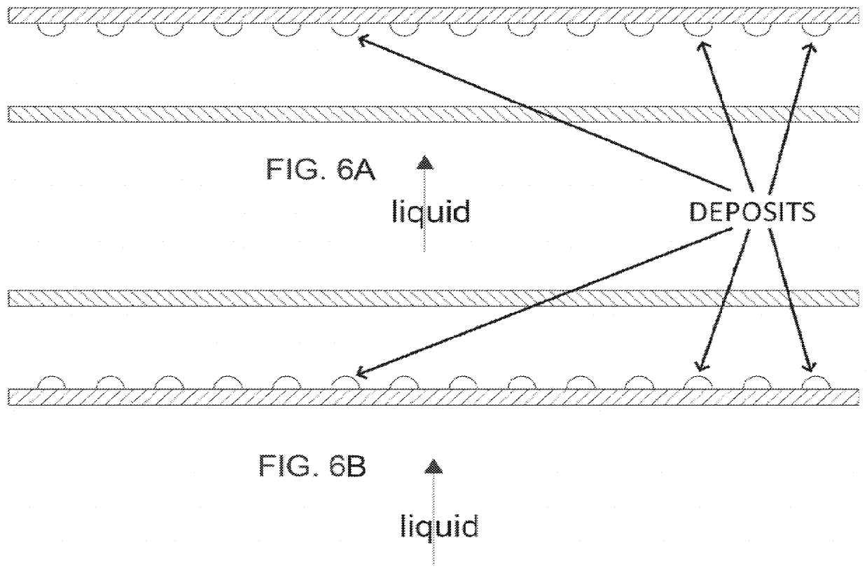

When only two layers are present in the multi-layer absorbent product (FIGS. 6A and 6B), and one layer is a layer of the textured plastic yarn with uniformly spaced deposits of SAP then the other layer is either a liquid permeable layer or a liquid impermeable layer. The liquid permeable layer or liquid impermeable layer can be any liquid permeable or liquid impermeable layer that is known and used in the art of either textile materials, woven, non-woven or knitted, or another plastic yarn that can be textured or smooth. If another plastic yarn is used as the second layer, the liquid permeability can be achieved by perforating the plastic yarn with small, penetrating holes. The size, shape and density of the holes can vary to adjust the liquid permeability or impermeability, for example, to allow for water vapor penetration through the second, upper layer without SAP deposits, while blocking or minimizing liquid water penetration through this layer. In a similar manner, the first layer with uniformly spaced SAP deposits can also be partially water-permeable, for example, by perforating it with small penetrating holes, that will allow water vapor penetration but will block or minimize liquid water permeability.

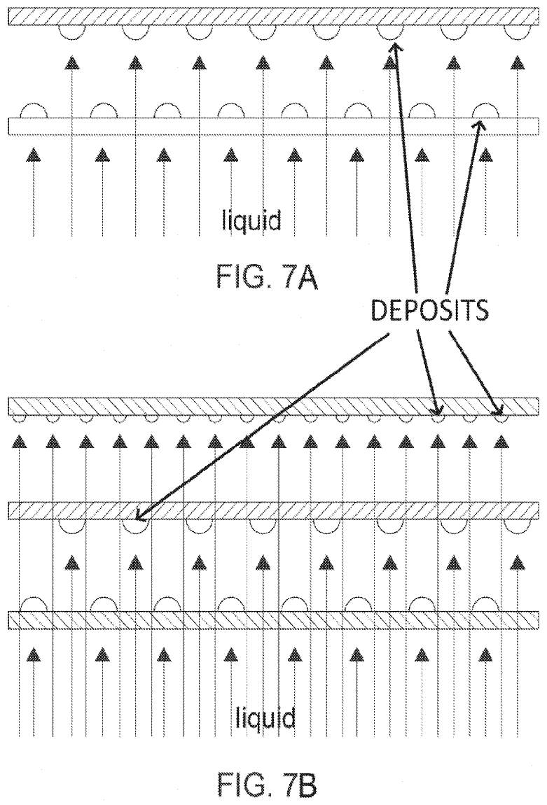

When only two layers are present in the multi-layer absorbent product (FIGS. 6A and 6B), and one layer is a layer of the textile material with uniformly spaced deposits of SAP, then the other layer is either a liquid permeable layer or a liquid impermeable layer. The liquid permeable layer or liquid impermeable layer can be any liquid permeable or liquid impermeable layer that is known and used in the art of absorbent products such as diapers, feminine hygiene absorbent pads, panty liners, cleaning wipes, household or institutional cleaning or maintenance appliances, hand wipes, hand towels, personal, cosmetic or sanitary wipes, baby wipes, facial tissues, hygienic absorbent pads, panties, wound dressings and the like. In the case of a two-layer absorbent product, the liquid permeable layer may be another layer (a second layer) of the textile material with uniformly spaced deposits of SAP. Preferably, the two layers of textile material with uniformly spaced deposits of SAP thereon are arranged where the top surface of the second layer is placed against the top surface of the first layer such that the uniformly spaced deposits on the top surfaces of the first and second layers do not overlap and contact each other (FIG. 7A). Thus, this arrangement of the two layers of textile material with uniformly spaced deposits of SAP provides a complementary design that avoids a gel blocking effect, where the swelling of the SAP deposits from absorption of liquid into a gel would undesirably close off (block) areas of the layer, such as the spaces between deposits, thereby allowing liquid to pass through to the next layer of uniformly spaced deposits for further absorption.

In addition to the two layers of the multi-layer absorbent product based on textile material of the present invention as described above, there may be one or more additional liquid permeable layers. At least one of these additional liquid permeable layers can be a further layer of nonwoven, woven or knitted textile material having a top surface with uniformly spaced deposits of SAP. The more layers of the textile material with uniformly spaced deposits of SAP in the multi-layer absorbent product, the more the total amount of SAP is available for absorbing liquids. For small amounts of liquid/fluids, such as in feminine care products for light menstrual flow, one layer with SAP deposits in the multi-layer absorbent product may be sufficient. However, where there is more fluid discharge that needs to be absorbed, such as in diapers, there can be three or even four layers with SAP deposits to provide a higher total amount of SAP available for greater liquid absorbancy. Having the deposits of SAP on different layers can help avoid a gel blocking effect by limiting the add-on weight of the deposits of SAP on a single layer of textile material. Another feature in optimizing high total amount of SAP deposits, total absorbance and absorbance speed for the multi-layer absorbent product of the invention is to have more layers that have SAP deposits but with different deposit sizes in the different layers and with relative positioning of the deposits in the different layers to avoid gel blocking effects. See, for example, FIG. 7B, where different size deposits on different layers are positioned relative to each other. By avoiding gel blocking effects, the maximum amount of SAP in the layers of the multi-absorbent product of the invention can absorb the maximum amount of liquid in the minimum amount of time.

The superabsorbent layer of nonwoven, woven or knitted textile material with uniformly spaced deposits of SAP is lightweight and thin and can be used in a feminine hygiene product marketed for the first and last day of the monthly menstrual period to provide a thin airable soft and skin friendly solution for what desirable and advantageous in such thin feminine hygiene products, with the added benefit that the presence of the natural polymer crosslinked to synthetic polymer in the SAP provides biodegradability.

Conventionally, the process for producing absorbent products, such as diapers and feminine hygiene pads, involves randomly distributing and mixing superabsorbent polymer in granulated powder form inside a fluffy pulp media to create a fluffy think pulp layer containing the granulated SAP as a thick absorbent core. This absorbent core is then wrapped in nonwoven or other wrapping layers to further create a thick wrapped absorbent core, where the wrapped absorbent core is cut into certain sizes. The sized absorbent core is integrated into additional layers of nonwoven/other material wrapping. In the conventional process, the crosslinking to form the SAP is done “offline” and then mixed with the fluffy pulp media. By contrast, the present invention provides a process where the polymer syrup solution is first applied to the textile material and then crosslinked (self-crosslinked) “onboard” the textile material as opposed to “offline”.

The present invention thus provides a process for producing the superabsorbent layer based on textile material that can be integrated into a multi-layer absorbent product such as diapers and feminine hygiene pads with added benefits, including:

### Benefits for the Hygiene Manufacturers, Such as:

Thinner hygiene products—no fluff is needed;
Lighter hygiene products—simpler design of the product and less “packaging”;
More “airable”—breathable Hygiene products;
Potentially eliminating absorbent core manufacturing—no core absorbent production
bottle neck and hence faster production;
Eliminating work environment hazards such as (1) SAP dust (2) SAP particles falling from the vibrating machines and causing slippery floor environment (both are known as major concerns for manufacturers)—SAP deposits stuck on the textile material substrate according the present invention will not fall apart or fall off the textile material; and
Option to offer a more biodegradable solution.

### Benefits for the Nonwoven Manufacturers, Such as:

New product functionality for nonwoven manufacturer—from “smart packaging for SAP” to “functional packaging”—packaging that also take part in the core functionality of the hygiene product—to absorb liquid
Allows the nonwoven industry to take a larger share in the hygiene value chain by providing additional functionality

The process for preparing the layer of nonwoven, woven or knitted textile material or textured plastic yarn with uniformly spaced deposits of a natural polymer crosslinked to a synthetic polymer used in the multi-layer absorbent product according to the present invention involves depositing on a nonwoven, woven or knitted textile material or on a textured plastic yarn (as substrate having a network of fibers or pores and pore walls) uniformly spaced deposits of a polymeric composite aqueous solution in the form of a thick and sticky syrup for use in improving water absorbency of the textile material or creating water absorbency of the plastic yarn. The polymeric composite solution includes a natural polymer and a synthetic polymer having monomers bearing carboxylic acid or carboxylic acid anhydride groups, where the natural and synthetic polymers in the composite solution are capable of undergoing self-crosslinking under controlled conditions of temperature and time and in the absence of non-polymeric crosslinking agent. The concentration of the combination of natural and synthetic polymers is in a range of 20-40% w/w of the polymeric composite solution. The textile material or textured plastic yarn having uniformly spaced deposits thereon are dried and then thermally treated (heat curing) under controlled conditions of temperature and time so that the natural and synthetic polymers in the deposit undergo self-crosslinking in the absence of non-polymeric crosslinking agent in the deposits to form SAP as a continuous polymer network that covers the fibers or pores and pore walls underneath the deposit and interpenetrates the network of fibers or pore walls acting as a scaffold to interconnect the SAP polymer network with the fiber network or network of plastic pore walls.

In the depositing step, the uniformly spaced deposits are deposited (“printed”) on a continuous roll of nonwoven, woven or knitted textile material or on textured plastic yarn preferably by rotary screen printing with a perforated cylinder in a 3D dot printing device. The 3D printing of polymer syrup as uniformly spaced deposits on a continuous roll of the textile material substrate or of the textured plastic yarn is preferably done with equipment in which rotary screen printing with a perforated cylinder is integrated with downstream continuous processing such as a downstream oven for drying and a heat curing thermal treatment of the deposits.

After the thermal treating step to self-crosslink the natural and synthetic polymers in the deposits, individual sheets of textured plastic yarn or of nonwoven, woven or knitted textile material with the uniformly spaced deposits can be cut from the continuous roll and used as a superabsorbent layer for assembly with other layers into the multi-layer absorbent product according to the invention. Alternatively, the multi-layer absorbent product may be assembled from continuous rolls of the different layer materials and then cut. The cutting and/or assembly may be done at a different facility from the 3D printing and thermal treatment.

The textured plastic yarn or nonwoven, woven or knitted textile material used in the process for the depositing step (i.e., prior to depositing) has a density between 20 to 150 g/m², preferably 20 to 60 g/m², more preferably 20 to 30 g/m².

After being thermally treated in the present process, the weight of the deposits relative to the textile material or to the plastic yarn is in a range of 100% to 600%, preferably 200% to 600%, more preferably 400% to 600%.

The deposits printed on the textile material substrate are uniformly spaced, with the spacing in a range of 1 to 7 mm, preferably 1 to 6 mm, more preferably about 2 mm and preferably printed as half domes, which become circular dots on the surface of the textile substrate when dried and thermally treated (FIG. 5B), or printed in a rectangular shape. It would be appreciated by those in the art that the printing of the deposits could be of any shape (or color by introducing a coloring agent) that may be desirable, such as from a functional or aesthetic perspective. One only needs to create a cylinder for rotary screen printing that has the desired shape or shapes (may be multiple shapes present).

The synthetic polymer (SP) used for self-crosslinking to the natural polymer in the deposit has monomers bearing carboxylic acid or carboxylic acid anhydride groups and is preferably selected from linear or branched graft homo- or copolymers made from vinyl acidic monomers such as acrylic acid, maleic anhydride, itaconic anhydride and similar, optionally in association with other types of vinylic monomers that do not necessarily contain carboxylic acid functions.

Other preferred embodiments of the synthetic polymer are a copolymer based on maleic anhydride and/or maleic acid, preferably a copolymer of styrene maleic anhydride (SMA), copolymer of isobutylene and maleic anhydride (e.g., commercially available copolymers sold under the tradename ISOBAM™) or copolymer of methyl vinyl ether and maleic acid (e.g., commercially available copolymers sold under the tradename GANTREZ™).

Preferably, the synthetic polymer is poly(decyl vinyl ether-alt-maleic anhydride), poly(ethyl vinyl ether-alt-maleic anhydride), poly(maleic acid-co-propene), poly(n-butyl vinyl ether-alt-maleic anhydride), poly(octadecene-alt-maleic anhydride), poly(propylene-alt-maleic acid) or poly(maleic acid-co-dodecyl methacrylate).

The synthetic polymer (SP) may be a commercially available polymer, having the following characteristics:

a) linear copolymers or branched graft homo- or copolymers that contain vinyl acidic monomers as: acrylic acid, maleic anhydride, itaconic anhydride and similar in association or not with other types of vinylic monomers which does not contain carboxylic groups.

b) content of total free carboxylic groups from 0.009-0.0095 mol/gram to 0.01-0.015 mol/grams

c) Free carboxylic groups of synthetic polymer, SP are in salt state, corresponding to a degree of neutralization between 49-99%, preferable between 60-95%, and most preferable between 65-90%;

d) The salt state of synthetic polymer is obtained by using strong inorganic base substances such as hydroxides, bicarbonates or carbonates of lithium, sodium, potassium or ammonium, preferred hydroxides of lithium, sodium, potassium or ammonium.

e) The average molecular weight of synthetic polymer, SP is from 50 kDa to 1,000 kDa, preferably from 100 kDa to 750 kDa, and most preferably from 150 kDa to 500 kDa.

The natural polymer (NP) used for self-crosslinking to the synthetic polymer in the deposit is a biopolymer selected from polysaccharides such as cellulose, alginate, dextran, chitosan, and the like; polyesters and lignin (in native forms or modified by chemical or enzymatic hydrolysis); guar; starch; water soluble phospholipids such as lecithin; polypeptides or proteins such as gelatin, albumin, soybean protein, collagen, collagenic biopolymer, collagen hydrolysate, and casein. The natural polymer has amino and/or hydroxyl groups capable of cross-linking to COOH groups in said synthetic polymer under high temperature conditions and for selected periods of time.

The natural polymer NP is preferably water-soluble phospholipids such as lecithin; polypeptides or proteins such as gelatin, albumin and the like; or polysaccharides such as cellulose, alginate, dextran, chitosan, and the like, that have at least one of the following characteristics:

a) average molecular weight from 5 to 100 kDa, preferably from 25 to 50 kDa, more preferably from 75 to 100 kDa.

b) free chemical groups such as amine or hydroxyl in a relative concentration from 0.001 to 0.01 mole/grams NP.

c) hydrophilic, capable of forming hydrogels in aqueous environment and capable of integration within the textile fibers.

According to preferred embodiments, the biopolymer used in the invention has the capability of cross-linking due to its NH₂ or OH groups that crosslink to COOH groups in the synthetic polymer under high temperature conditions and for selected periods of time, to form ester and amide bonds between polymers skeletons. Furthermore, the biopolymer activates the biodegradation of the textile impregnated with composite polymer solution of SP and NP.

The polymeric composite aqueous solution used in the process according to the present invention for preparing the layer of nonwoven, woven or knitted textile material or the layer of textured plastic yarn with uniformly spaced deposits of a natural polymer crosslinked to a synthetic polymer which is part of the multi-layer absorbent product can be prepared as follows:

a) preparation of alkaline base solution

b) preparation of aqueous solution of synthetic polymer (SP) in acidic form, which is treated afterwards with the alkaline solution from (a) to obtain the salt form, at a concentration of between 1% and 10%, preferably between 2 and 5% by weight,

c) preparation under heating of aqueous solution of natural polymer (NP) at a concentration between 1% and 10%, preferably between 2 and 5% by weight

d) mixing under heating and stirring the SP solution obtained in (b) in salt form, with the NP solution obtained in (c) to obtain aqueous stable composite solution of polymers suitable to be used as impregnation mass that confers high water absorbance to textile materials

and optionally

e) adding to the aqueous composite solution obtained in (d) at least one auxiliary material selected from the list of plasticizers, surface agents, deodorants, perfume and preservatives.

The polymers composite solution used is a stable solution of polymer materials SP and NP, without phase separation under conditions of storage or ulterior processing and may further contain at least one compatible additive used in the textile industry such as plasticizers, dyes, antibacterial agents, surfactants, deodorants, perfume, preservatives, etc., in quantities that are correlated with other properties than absorbency and without affecting the water absorptive performance or biodegradability of the textile material. The polymers composite solution is suitable for impregnation into fabrics and also suitable for self-crosslinking under controlled conditions of temperature and time.

Without being bound to theory, the polymer composite solution can interpenetrate within the textile fibers or the textured plastic yarn pore walls and self-crosslink under conditions of thermal treatment at temperatures between 100 to 250° C. and during periods of preferably between 2 to 30 minutes.

Impregnation of the textile material is done using any equipment known in art, for example spray devices; foulard; roll coating; reverse roll coating; knife devices etc., preferably using rotary screen printing with a perforated cylinder in a 3D dot printer device.

### EXAMPLES

### Test Methods

1. Characterization of polymer solutions containing synthetic polymer, natural polymer and auxiliary
The viscosity of the solution was evaluated using a viscosimeter ViscoStar Plus, Fungilab, Spain using a volume of solution correlated to the type of spindle L1 at 25° C. temperature.
The stability of the polymer solution used for impregnation was assessed to be stable or unstable if the analyte solution showed sediment after centrifugation of a volume of 25 ml solution at 5000 rpm for 30 minutes. Tests were performed with a laboratory centrifuge GLC-2B Sorvall, Thermo Scientific at ambient temperature.

### 2. Free Absorbency

The following measurements are made:

Mtex, [grams], mass of the dried textile material used for impregnation by weighing at a semi-analytical balance

Mtwstart, [grams]—mass of wet starting material by weighing the sample

Mtid-[grams]—mass of drained textile material after impregnation

MIC-[grams]—mass of dried polymeric composite material found in the textile after impregnation evaluated by the formula:

MIC=Mtex*IMD/100,grams

The degree of impregnation of a textile material is established using the formula:

[IMD]dry=MIC/(Mtex+MIC)*100,%

### Absorbency Evaluation of Textile Materials:

2.1 Absorbency of textile material non-impregnated Q1 consists of introducing the textile sample, M-tex into a 150 ml volume of liquid to which the absorbance value is desired so that the entire surface of the textile material is covered by the liquid and the contact is maintained without shaking for 60 minutes. Ulterior, the material is removed from the liquid, it hangs in a vertical position to drain excess liquid for 15 minutes. The drained textile material sample is weighed and the value obtained is recorded as M-tex-wet.

Absorbency of non-impregnated textile material Q1 is obtained by using the formula:

Q1=(M-tex wet-M-tex)/M-tex,(g/g)

### 2.2 Absorbency of Impregnated Textile Material Q2

The textile sample is weighted at technical balance obtaining the value Mtex. Then is impregnated with a chosen mass of liquid:distilled water, an impregnating solution or other type of solution by using a laboratory spray device. The wet material thus obtained is weighted again and the value is Mtwstart. If this value is higher than the impregnation degree (IMD)-pre-established the wet sample is subdue to a squeezing process with the aid of a glass rod to eliminate the excess of impregnation liquid so that it finally is obtained a wet sample with the mass Mtid. Next, the wet sample is kept for 30 minutes, in a closed glass beaker to avoid evaporation of the liquid.

The absorbency of textile material sample is obtained with formula:

Q2−TEXIC=[Mtid−(Mtex+MIC)]/(Mtex+M IC),(g/g)

### 2.3. Relative Absorbency

RQ represents the ratio between absorbency of an impregnated textile material Q2 and absorbency of the same textile material non-impregnated Q1.

Relative absorbency is calculated by using the formula:

[RQ]1=Q2/Q1or

[RQ]2=[(RQ1)−1]*100,(%)

Having now generally described the invention, the same will be more readily understood through reference to the following example which is provided by way of illustration and is not intended to be limiting of the present invention.

### Example 1

Stock solutions of synthetic polymer, natural polymer and inorganic base are prepared as follows:

### a) Stock of Synthetic Polymer Solution:

42.6 g of SMAc styrene/maleic acid copolymer [prepared as in U.S. Pat. No. 7,985,819] in powder form having an 8% moisture content with an average molecular weight of 450,000 Da containing 0.0091 mol/g free carboxylic groups together with 358 g of demineralized water were added to a mixing vessel with agitation and the content was mixed for 1 hour at 80° C. to complete dissolution of the synthetic polymer and is end by cooling the polymer solution to 40° C. Finally, 400 g of SMAc polymer solution of 10% by dry weight is obtained.

### b) Stock of Sodium Hydroxide Solution

Is prepared 400 g of NaOH solution of 10% dry weight (from 98.9% pure sodium hydroxide pellets) and demineralized water using a mixing vessel with agitation fitted with a heating-cooling jacket.

### c) Stock of Natural Polymer Solution

400 g of gelatin type A solution with 200 Bloom and 14% moisture content (as natural polymer-NP) of 10% dry weight were prepared by dissolving 46.5 g of natural polymer in 354 g of demineralized water using a mixing vessel by stirring with a rotor speed not higher than 60 rpm, during a period of 1 hour at a temperature of 40° C. so as to ensure the perfect homogenization of the solution.

Further, the 3 types of solutions prepared above were used to prepare 3 sets of composite solutions by diluting with demineralized water the stock solutions as follows:

Set-SOL1 without gelatin made up of 12 solutions of 3% concentration in which the synthetic polymer has a different degree of neutralization between 0% and 110%.

Set-SOL-2 containing gelatin in proportion of 10% to SMAc consisting of 12 solutions of 3% concentration in which the synthetic polymer has a different degree of neutralization between 0% and 110% and Set-SOL-3 which contains gelatin in a proportion of 30% to SMAc consisting of 12 solutions of 3% concentration in which the synthetic polymer has a different degree of neutralization ranging between 0% and 110%.

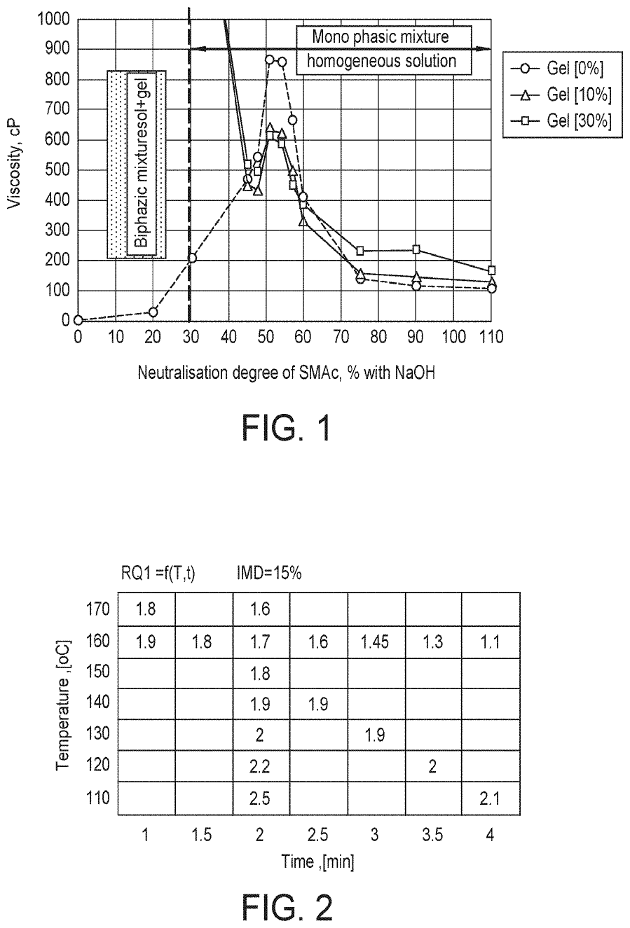

All composite solutions corresponding to the three sets were characterized in terms of their viscosity and stability in the sense that the sample is unstable if it contains the insoluble phase and that the sample is stable if it is a homogeneous solution without the insoluble phase. The results obtained are presented in Table 1 and FIG. 1.

TABLE 1
Influence of the degree of neutralization of the synthetic
polymer SPS and of the natural polymer NP content
(as gelatin type A with 200 Bloom) in the composition
SP: NP) on solutions' viscosity [h, (cP)] of 3% by dry weight.

| Neutralization | Gel [0%] | Gel [10%] | Gel [30%] |
|---|---|---|---|
| degree of SP, [%] | η; [cP] | η; [cP] | η; [cP] |
| 0 | 4.3 | 2000 | 2000 |
| 2 | 28.7 | 1900 | 1900 |
| 3 | 209.6 | 1800 | 1800 |
| 45 | 473.6 | 454.3 | 519.2 |
| 48 | 543.7 | 432.8 | 491.3 |
| 51 | 867.5 | 634.1 | 612.6 |
| 54 | 858 | 624.8 | 589.2 |
| 57 | 667.1 | 496.7 | 452.2 |
| 60 | 409.5 | 330.6 | 386.8 |
| 75 | 141.7 | 160 | 231.4 |
| 90 | 120.1 | 147.3 | 234.8 |
| 110 | 110.4 | 129.5 | 165.9 |

From FIG. 1 it is noted that the presence of the natural polymer leads to a decrease in the viscosity of the synthetic polymer if the degree of neutralization of the synthetic polymer is between 30-60%. If the degree of neutralization is greater than 60%, the presence of the biopolymer determines the increase in the viscosity value of the composite solutions. From the point of view of the stability of the composite solutions, this is dependent on the degree of neutralization of the synthetic polymer. It has been noted that composite systems containing SMAc and gelatin are stable if the degree of neutralization of the synthetic polymer is greater than 30%.

### Examples 2 to 5

In these examples, is presented the absorbency value to demineralized water (conductivity 2 micro S) of some nonwoven fabrics containing viscose fiber with density of 50 g/m² with different impregnation degree with polymeric solutions' concentration of 1% by weight containing SMAc (mentioned in Example 1) with neutralization degree of 59% generated with NaOH and various values of gelatin content and which additionally contains 1.5% by dry weight to the content of the polymeric composition.

The Experiments were Conducted as Follows:

Textile materials having a mass of 0.5 g were impregnated with composite solutions prepared according to the method described in Example 1 by spraying with a laboratory device so as finally to obtain the impregnation degree pre-established, IMD, to the dry mass of the sample of dried fabric material. Further, the wet textile samples were placed in a hanging state in an oven with air circulation, preheated to 180° C. and are maintained at this temperature for 4 minutes. Finally, the sample was removed from the oven and allowed to cool at ambient temperature. Then the textile material was subjected to the absorbency test according to the methodology described in the chapter “Test methods”.

Experimental conditions and results are shown in Table 2.

TABLE 2
The influence of gelatin content in the polymeric solution for impregnation and the impregnation
degree on the absorbency of impregnated nonwoven

| Example | | Gelatin in | IMD, | Q₂-texic, | |
|---|---|---|---|---|---|
| cod | [Q-tex]_{water} | Composite, % | % | (g/g) | RQ₁ |
| Exp-2 | 9.19 | 7.6 | 8 | 15.79 | 1.71 |
| Exp-3 | 9.19 | 9.7 | 10 | 12.08 | 1.39 |
| Exp-4 | 9.19 | 13.5 | 8 | 14.07 | 1.53 |
| Exp-5 | 9.19 | 17.6 | 12 | 15.49 | 1.68 |

### Examples 6-12

In these examples are presented the impregnation of a textile material consisting of viscose fibers with a density of 50 g/m² using a solution of synthetic polymer SMAc with neutralization degree of 64% done by using NaOH, 3.6% gelatin to the polymeric composite at degree of impregnation of 20% with thermal treatment of 200° C. for 100 seconds using the same oven with air circulation mentioned in the previous examples. Finally, the absorbency of the textile sample was evaluated against various liquid media represented by simulated fluid secreted by the human body (Margareth R. C. Marques et al, Simulated Biological Fluids with Possible Application in Dissolution Testing, Dissolution Technologies AUGUST 2011, 1)

The results are presented in Table 3 below.

TABLE 3
Influence of the liquid medium's composition on the absorbency of textile
materials impregnated with polymeric composites containing synthetic
polymer and gelatin

| Examples | | M-tex | IMD | Q₁ | Q₂ tex |
|---|---|---|---|---|---|
| code | Liquid media | [g] | dry % | [g/g] | [g/g] |
| Exp-6 | Distillated water | 0.5 | 15 | 29.03 | 50.48 |
| | 0.0002 mS | | | | |
| Exp-7 | Tap water | 0.5 | 15 | 16.20 | 28.17 |
| | 4 mS | | | | |
| Exp-8 | Salt water 0.9% | 0.5 | 15 | 5.89 | 10.24 |
| | 15.4 mS | | | | |
| Exp-9 | Bovine Milk (3%) | 0.5 | 15 | 5.11 | 8.88 |
| Exp-10 | SimulatedHuman | 0.5 | 15 | 2.81 | 4.89 |
| | Sweat⁽¹⁾ | | | | |
| Exp-11 | SimulatedWound | 0.5 | 15 | 6.28 | 10.92 |
| | Fluids⁽¹⁾ | | | | |
| Exp-12 | Simulated Saliva⁽¹⁾ | 0.5 | 15 | 7.63 | 13.26 |

### Example 13

In this example is presented the influence of the temperature and the time of the thermal treatment at what is subjected a sample of textile material with a density of 50 g/m² in order to obtain an absorbent textile material on the relative absorption RQ1. The results are presented in FIG. 2.

The data obtained show that the thermal treatment of the impregnated fabrics must be in such manner as to obtain the highest value for absorbency. The lower values of the maximum result either because of a low crosslinking degree or because the degree of crosslinking of the polymeric composite is too high.

### Example 14

This example shows the influence of the type of fiber from which the fabric is done subjected to impregnation.

For this purpose, a composite polymer solution having the chemical composition coinciding with Exp-2 was used.

Have been used textile materials from polypropylene fiber (PP-fiber), polyester fibers (PET-fiber) viscose fibers (Viscose-fiber).

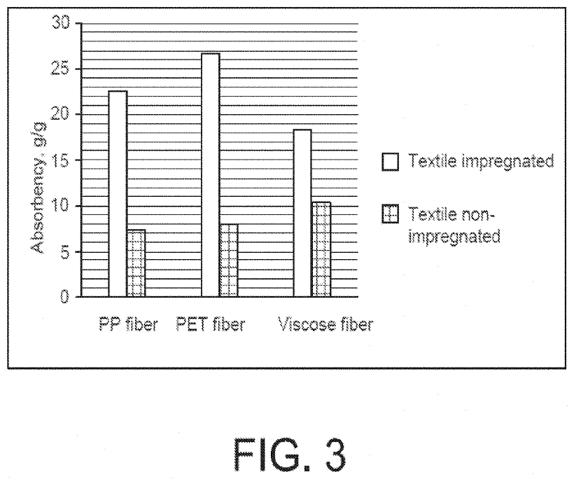

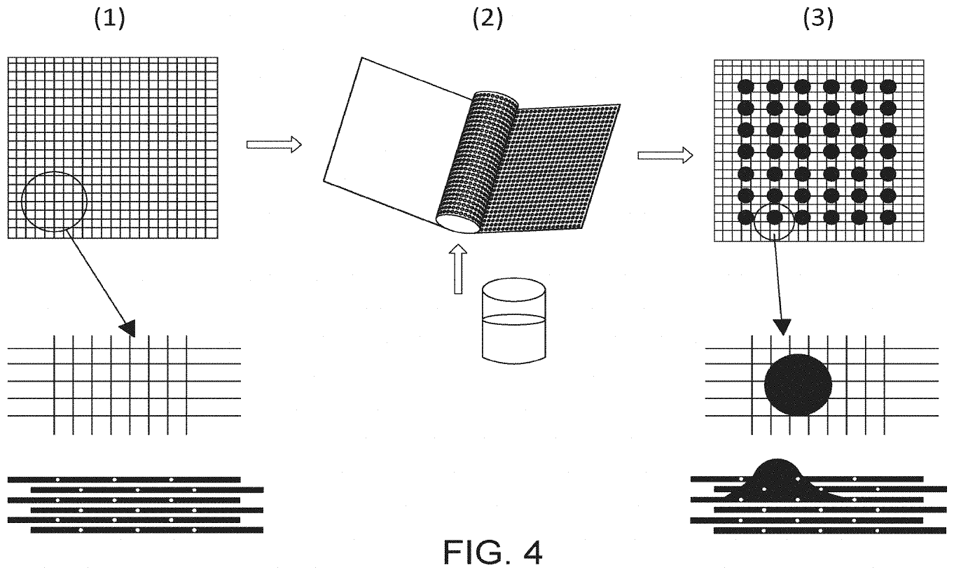

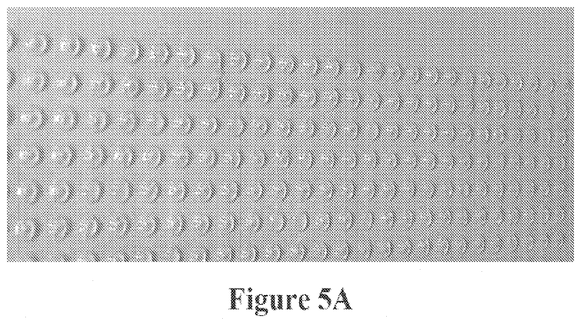

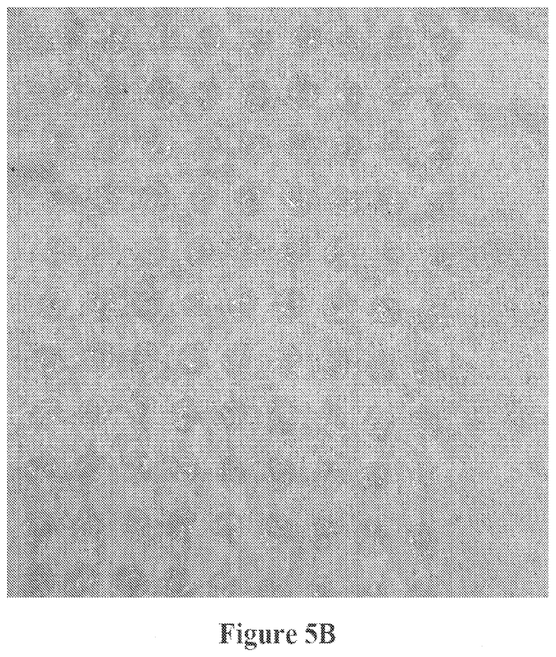

The results obtained are presented in FIG. 3.

### Examples 15-16

In these examples is presented the influence of the type of biopolymer used for the preparation of composite material according to the invention on the absorbency of a textile material using the preparation technology for the composite from Example land the methodology of impregnation of the textile material from Example 3.

Instead of gelatin have been used gum guar (G4129 Sigma Aldrich) and soluble starch (S9765 Sigma Aldrich).

TABLE 4
Influence of the biopolymer type (carbohydrates) on the absorbency
of textile materials impregnated with polymeric composites containing
synthetic polymer and biopolymer

| | Natural | Natural | | |
|---|---|---|---|---|
| | polymer | polymer in | | Q₂-texic, |
| Example cod | type | composite, % | IMD, % | (g/g) |
| Exp-15 | Guar | 1.8 | 19 | 16.96 |
| Exp-16 | Starch | 2.3 | 28 | 20.44 |

### Example 17

TABLE 5
Comparison of dry weight, absorbency in tap water and saline solution in
nonwoven polypropylene (PP) and printed SAP on the same nonwoven PP

| | | | Printed SAP on nonwoven | |
|---|---|---|---|---|
| | Nonwoven | | PP (175% add-on) | |
| | PP 22 [g/m²] | | | Add-on |
| | gr or gr/gr | Reference | gr or gr/gr | relative to Ref. |
| Dry weight [gr] | 0.4 | 100% | 1.1 | 275% |
| Absorbency tap water | 3 | 100% | 63 | 2100% |
| [gr/gr]* | | | | |
| Absorbency saline [gr/gr]* | 3 | 100% | 21 | 700% |

*Measured according to EDANA NW5P 010.1.R0. Saline = 0.9% NaCI solution, tap water = San Benedett mineral water. This sample provides 21 times the absorbency in tap water and 7 times the absorbency in saline relative to the regular product (nonwoven polypropylene (PP))

The polymer composite solution comprises synthetic polymer in the form of salt (SP), natural polymer (NP), additives (A) and water (W).

The ratio between a textile material TEX (before treatment) to impregnation composition IC [IC=SP+NP+A] is from 85:25 to 99:1% by dry weight.

The ratio between the synthetic polymer (SP) in salt form to natural polymer (NP) SP/NP is from 70:30 to 95:5% by dry weight.

The ratio of additives (A) to polymers A/(SP+NP) is from 0.5 to 5% dry weight.

Water content in the composite polymer solution is from 79 to 99% by weight.

Further processing of the wet textile material depends of initial density of material and of impregnation degree so chosen. For example:

a) if the density of the textile material has been initially higher than 70 g/m², then the wet textile material is first dried in a stream of hot air at temperature of 50-90° C., preferable at temperature of 55-85° C., and most preferable at temperature of 60-80° C., so the solid composite (textile+composite polymer) resulted to have a humidity content less than 12% by weight, preferable less than 10% by weight, and most preferable less than 8% by weight and then the supplementary thermal treatment in hot air steam at temperature of 90-180° C., preferable at temperature of 100-170° C., and most preferable at temperature of 110-160° C., during a period of 5-180 minutes, preferable 10-150 minutes, and most preferable of 15-120 minutes;

b) if the density of textile material has been initially less than 70 g/m² the wet textile material is subdue to a single thermal treatment at temperature of 100-150° C., during 180-300 seconds, preferable at temperature of 140-180° C. during 60-180 seconds and more preferable at temperature of 200-250° C. during 30-120 seconds.

Following the thermal treatment the textile material is let to cool at room temperature and in the end is packed.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Margareth R. C. Marques, Raimar Loebenberg, and May Almukainzi, Simulated Biological Fluids with Possible Application in Dissolution Testing Dissolution Technologies, AUGUST 2011, 1

U.S. Patent Documents

| | | |
|---|---|---|
| 2,988,539 | Cohen,et al. | Jun. 13, 1961 |
| 3,393,168 | Johnson, et al. | Jul. 16, 1968 |
| 3,514,419 | Durlow, et al. | May 26, 1970 |
| 3,926,891 | Gross,e t al. | Dec. 16,1975 |
| 3,980,663 | Gross; James R. | Sep. 14, 1976 |
| 3,983,271 | Pangle, Jr., et al. | Sep. 28, 1976 |
| 4,057,521 | Gross; James R | Nov. 8, 1977 |
| 4,066,584 | Allen, et al. | Jan. 3, 1978 |
| 4,155,957 | Sasayama; Hiroharu | May 22, 1979 |
| 4,210,489 | Markofsky; Sheldon B. | Jul. 1, 1980 |
| 4,242,408 | Evani, et al. | Dec. 30, 1980 |
| 4,320,040 | Fujita, et al. | Mar. 16, 1982 |
| 4,418,163 | Murakami, et al. | Nov. 29, 1983 |
| 4,537,590 | Pieniak, et al. | Aug. 27, 1985 |
| 4,540,454 | Pieniak, et al. | Sep. 10, 1985 |
| 4,573,988 | Pieniak, et al. | Mar. 4, 1986 |
| 4,676,784 | Erdman, et al. | Jun. 30, 1987 |
| 4,731,067 | Le-Khac; Bi | Mar. 15, 1988 |
| 4,855,179 | Bourland, et al. | Aug. 8, 1989 |
| 4,861,539 | Allen, et al. | Aug. 29, 1989 |
| 4,880,868 | Le-Khac; Bi | Nov. 14, 1989 |
| 4,962,172 | Allen, et al. | Oct. 9, 1990 |
| 4,963,638 | Pazos, et al. | Oct. 16, 1990 |
| 5,183,707 | Herron, et al. | Feb. 2, 1993 |
| 5,280,079 | Allen, et al. | Jan. 18, 1994 |
| 5,413,747 | Akers, et al. | May 9, 1995 |
| 5,567,478 | Houben, et al. | Oct. 22, 1996 |
| 5,693,707 | Cheng, et al. | Dec. 2, 1997 |
| 5,698,074 | Barcus, et al. | Dec. 16, 1997 |
| 5,962,068 | Tsuchiya, et al. | Oct. 5, 1999 |
| 5,997,791 | Chou, et al. | Dec. 7, 1999 |
| 6,150,495 | Chou, et al. | Nov. 21, 2000 |
| 6,162,541 | Chou, et al. | Dec. 19, 2000 |
| 6,241,713 | Gross, et al. | Jun. 5, 2001 |
| 6,417,425 | Whitmore, et al. | Jul. 9, 2002 |
| 6,506,696 | Goldstein, et al. | Jan. 14, 2003 |
| 6,645,407 | Kellenberger, et al. | Nov. 11, 2003 |
| 6,689,378 | Sun, et al. | Feb. 10, 2004 |
| 6,773,746 | Bell; Otis Franklin | Aug. 10, 2004 |
| 7,300,965 | Weerawarna, et al. | Nov. 27, 2007 |
| 7,985,819 | Bucevschi et al | July 26,2011 |

1. A multi-layer absorbent product, comprising:
a first layer which is liquid permeable or liquid impermeable; and
a layer of (i) nonwoven, woven or knitted textile material comprising a network of fibers or (ii) textured plastic yarn comprising a network of surface pores and being disposed next to the first layer, said layer of textile material or textured plastic having a top surface with uniformly spaced super absorbent polymer (SAP) deposits of a synthetic polymer having monomers bearing carboxylic acid or carboxylic acid anhydride groups crosslinked to a natural polymer having amino and/or hydroxyl groups capable of crosslinking to the synthetic polymer in the absence of non-polymeric crosslinking agent on said network of fibers or surface pores, and said top surface with uniformly spaced deposits being disposed against a surface of the first layer,
wherein said SAP deposits of natural polymer crosslinked to synthetic polymer forms a continuous SAP crosslinked polymer network that covers the fibers or pore walls underneath the SAP deposits and interpenetrates the network of fibers or network of pores acting as a scaffold to interconnect the SAP crosslinked polymer network with the fiber or pore network, and
said layer of (i) nonwoven, woven or knitted textile material or (ii) textured plastic yarn with said SAP deposits have a water absorbency at least 10% higher than the absorbency of the same layer without said SAP deposits.

2. The multi-layer absorbent product of claim 1, wherein said layer is a layer of nonwoven, woven or knitted textile material comprising a network of fibers.

3. The multi-layer absorbent product of claim 2, wherein said layer of nonwoven, woven or knitted textile material with said deposits has a water absorbency at least 20%, at least 30%, at least 50%, at least 80%, or at least 100% higher than the absorbency of the same layer without said deposits, preferably at least 3 times higher or at least 4 times higher, more preferably at least 5 times higher.

4. The multi-layer absorbent product of claim 1, wherein said layer of nonwoven, woven or knitted textile material, or textured plastic yarn having said deposits is capable of absorbing at least 2 grams water per gram (g/g; gr/gr) of said layer, at least 5 gr/gr, at least 10 gr/gr, preferably at least 20 gr/gr, more preferably at least 30 gr/gr, at least 40 gr/gr, at least 60 gr/gr, most preferably at least 80 gr/gr or at least 100 gr/gr.

5. The multi-layer absorbent product of claim 1, wherein said layer of nonwoven, woven or knitted textile material without said deposits has a density between 20 to 150 g/m², preferably 20 to 60 g/m², more preferably 20 to 30 g/m², or wherein said layer of textured plastic yarn without said deposits has a density between 18 and 150 gr/m², preferably 18 to 77 gr/m².

6. The multi-layer absorbent product of claim 1, wherein the weight of said deposits relative to the textile material or textured plastic yarn is in a range of 100% to 600%, preferably 200% to 600%, more preferably 400% to 600%.

7. The multi-layer absorbent product of claim 1, wherein said deposits are uniformly spaced on said layer of nonwoven, woven or knitted textile material or textured plastic yarn, with the spacing in a range of 1 to 7 mm, preferably 1 to 6 mm, more preferably about 2 mm.

8. The multi-layer absorbent product of claim 1, wherein the textile material and network of fibers or the textured plastic yarn and network of pores is polypropylene.

9. The multi-layer absorbent product of claim 1, wherein said deposits are in the shape of circular dots or rectangles on the textile material or textured plastic yarn.

10. The multi-layer absorbent product of claim 1, wherein the synthetic polymer is preferably selected from linear or branched graft homo- or copolymers made from vinyl acidic monomers such as acrylic acid, maleic anhydride, itaconic anhydride and similar, optionally in association with other types of vinylic monomers that do not necessarily contain carboxylic acid functions.

11. The multi-layer absorbent product of claim 1, wherein said synthetic polymer is a copolymer based on maleic anhydride and/or maleic acid, preferably a copolymer of styrene maleic anhydride (SMA), copolymer of isobutylene and maleic anhydride or copolymer of methyl vinyl ether and maleic acid.

12. The multi-layer absorbent product of claim 1, wherein said natural polymer is a biopolymer selected from polysaccharides such as cellulose, alginate, dextran, chitosan, and the like; polyesters and lignin; guar; starch; water soluble phospholipids such as lecithin; polypeptides or proteins such as gelatin, albumin, soybean protein, collagen, collagenic biopolymer, collagen hydrolysate, and casein.

13. The multi-layer absorbent product of claim 1, wherein said natural polymer has amino and/or hydroxyl groups capable of cross-linking to COOH groups in said synthetic polymer under high temperature conditions and for selected periods of time.

14. The multi-layer absorbent product of claim 1, wherein said first layer is a liquid impermeable layer.

15. The multi-layer absorbent product of claim 1, wherein said first layer is a liquid permeable layer.

16. The multi-layer absorbent product of claim 15, wherein the liquid permeable layer is a second layer of said nonwoven, woven or knitted textile material having a top surface with said uniformly spaced deposits, and wherein the top surface of said second layer is disposed against the top surface of said first layer of said nonwoven, woven or knitted textile material such that the uniformly spaced SAP deposits on the top surfaces of said first and second layers do not overlap.

17. The multi-layer absorbent product of claim 14, further comprising one or more liquid permeable layers.

18. The multi-layer absorbent product of claim 17, wherein at least one of said one or more liquid permeable layers is a further layer of said nonwoven, woven or knitted textile material having a top surface with said uniformly spaced SAP deposits.

19. (canceled)

20. A process for preparing a layer of nonwoven, woven or knitted textile material or a layer of textured plastic yarn, with uniformly spaced deposits of a natural polymer crosslinked to a synthetic polymer in the multi-layer absorbent product of claim 1, comprising:
depositing on a nonwoven, woven or knitted textile material, which has a network of fibers, or on a textured plastic yarn, which has a surface network of pores, uniformly spaced deposits of a polymeric composite aqueous solution in the form of a syrup for use in improving water absorbency of the textile material or textured plastic yarn, the polymeric composite solution comprising a synthetic polymer having monomers bearing carboxylic acid or carboxylic acid anhydride groups and a natural polymer having amino and/or hydroxyl groups capable of crosslinking to the synthetic polymer, in which the natural and synthetic polymers in the composite solution are capable of undergoing self-crosslinking under controlled conditions of temperature and time and in the absence of non-polymeric crosslinking agent, wherein the concentration of the combination of natural and synthetic polymers is in a range of 20-40% w/w of the polymeric composite solution; and
thermally treating the textile material or textured plastic yarn having uniformly spaced deposits thereon under controlled conditions of temperature and time so that the natural and synthetic polymers in the deposit undergo self-crosslinking in the absence of non-polymeric crosslinking agent in the deposits to form a continuous SAP crosslinked polymer network that covers the fibers or pore walls underneath the SAP deposits and interpenetrates the network of fibers or pores acting as a scaffold to interconnect the SAP crosslinked polymer network with the fiber or pore network.

21. The process of claim 20, wherein, in the depositing step, the uniformly spaced deposits are deposited on a continuous roll of nonwoven, woven or knitted textile material or textured plastic yarn by rotary screen printing with a perforated cylinder, and from which individual sheets of the nonwoven, woven or knitted textile material or textured plastic yarn with uniformly spaced SAP deposits thereon are later cut.

22. The process of claim 21, further comprising, after the thermal treating step, a step of cutting individual sheets of nonwoven, woven or knitted textile material or textured plastic yarn with uniformly spaced SAP deposits from the continuous roll.

23. (canceled)

24. The process of claim 20, wherein, after being thermally treated, the weight of the deposits relative to the textile material is in a range of 100% to 600%, preferably 200% to 600%, more preferably 400% to 600%.

25-31. (canceled)

32. The multi-layer absorbent product of claim 1, wherein said layer is a layer of textured plastic yarn comprising a network of surface pores.

33-34. (canceled)

35. The multi-layer absorbent product of claim 32, wherein said layer of textured plastic yarn without said deposits has a thickness between 20 to 200 micrometers, preferably 20 to 80 micrometers.

36-45. (canceled)

46. The multi-layer absorbent product of claim 32, wherein said first layer is a layer permeable for water vapor but not for liquids.

47. The multi-layer absorbent product of claim 32, wherein said first layer is a liquid impermeable layer, perforated with holes that confer on the layer a certain degree of water vapor and liquid water permeability.

48. The multi-layer absorbent product of claim 14, further comprising one or more liquid impermeable layers.

49. The multi-layer absorbent product of claim 48, wherein at least one of said one or more liquid impermeable layers is a further layer of said textured plastic yarn having a top surface with said uniformly spaced SAP deposits.

50-62. (canceled)
